SEQUENCE LISTING (14 sequences)

SEQ ID NO: 1 - protein, 20 aa, Artificial Sequence
  Variant of the wildtype Akt Sequence
VELDPEFEPRARERTYSFGH

SEQ ID NO: 2 - protein, 20 aa, Artificial Sequence
  Variant of the wildtype Akt Sequence
VELDPEFEPRARERTYAFGH

SEQ ID NO: 3 - protein, 20 aa, Artificial Sequence
  Variant of the wildtype Akt Sequence
VELDPEFEPRARERTYDFGH

SEQ ID NO: 4 - protein, 20 aa, Artificial Sequence
  Variant of the wildtype Akt Sequence
VELDPEFEPRARERAYAFGH

SEQ ID NO: 5 - protein, 20 aa, Artificial Sequence
  Variant of the wildtype Akt Sequence
VELDPEFEPRARERDYAFGH

SEQ ID NO: 6 - DNA, 60 bp, Artificial Sequence
  Nucleic acid sequence encoding peptide inhibitor
gtngarytngayccngarttygarccnmgngcnmgngarmgnacntaygayttyggncay

SEQ ID NO: 7 - DNA, 60 bp, Artificial Sequence
  Nucleic acid sequence encoding peptide inhibitor
gtngarytngayccngarttygarccnmgngcnmgngarmgnacntaygcnttyggncay

SEQ ID NO: 8 - DNA, 60 bp, Artificial Sequence; identical to SEQ ID NO: 6
  Nucleic acid sequence encoding peptide inhibitor

SEQ ID NO: 9 - DNA, 60 bp, Artificial Sequence
  Nucleic acid sequence encoding peptide inhibitor
gtngarytngayccngarttygarccnmgngcnmgngarmgngcntaygcnttyggncay

SEQ ID NO: 10 - DNA, 60 bp, Artificial Sequence
  Nucleic acid sequence encoding peptide inhibitor
gtngarytngayccngarttygarccnmgngcnmgngarmgngaytaygcnttyggncay

SEQ ID NO: 11 - protein, 524 aa, Homo sapiens
MSPIDPMGHHHHHHGRRRASVAAGILVPRGSPGLDGICSIEEFTMSDVAIVKEGWLHKRGEYIKTWRPRYFLLKNDGTFIGYKERPQDVDQREAPLNNFSVAQCQLMKTERPRPNTFIIRCLQWTTVIERTFHVETPEEREEWTTAIQTVADGLKKQEEEEMDFRSGSPSDNSGAEEMEVSLAKPKHRVTMNEFEYLKLLGKGTFGKVILVKEKATGRYYAMKILKKEVIVAKDEVAHTLTENRVLQNSRHPFLTALKYSFQTHDRLCFVMEYANGGELFFHLSRERVFSEDRARFYGAEIVSALDYLHSEKNVVYRDLKLENLMLDKDGHIKITDFGLCKEGIKDGATMKTFCGTPEYLAPEVLEDNDYGRAVDWWGLGVVMYEMMCGRLPFYNQDHEKLFELILMEEIRFPRTLGPEAKSLLSGLLKKDPKQRLGGGSEDAKEIMQHRFFAGIVWQHVYEKKLSPPFKPQVTSETDTRYFDEEFTAQMITITPPDQDDSMECVDSERRPHFPQFSYSASSTA

SEQ ID NO: 12 - protein, 22 aa, Artificial Sequence
  Biotinylated peptide
EELSPFRGRSRSAPPNLWAAQR

SEQ ID NO: 13 - protein, 14 aa, Mus musculus
ARKRERTYSFGHHA

SEQ ID NO: 14 - protein, 18 aa, Artificial Sequence
  Variant of the wildtype Akt sequence
TTYADFIASGRTGRRNAI

(19) United States
(12) Patent Application Publication
(10) Pub. No.: US 2004/0038883 A1
(43) Pub. Date: Feb. 26, 2004

(54) Peptide inhibitors of Akt and uses thereof

(76) Inventors: Yan Luo, Lake Bluff, IL (US); Vincent L. Giranda, Gurnee, IL (US); Paul L. Richardson, Libertyville, IL (US); Richard A. Smith, Lake Bluff, IL (US)

Correspondence Address: STEVEN F. WEINSTOCK, ABBOTT LABORATORIES, 100 ABBOTT PARK ROAD, DEPT. 377/AP6A, ABBOTT PARK, IL, 60064-6008, US

(21) Appl. No.: 10/365,348

(22) Filed: Feb. 12, 2003

Related U.S. Application Data
(63) Continuation-in-part of application No. 10/103,256, filed on Mar. 21, 2002.

(51) Int. Cl. A61K 38/17; C07K 14/47; C12P 21/02; C12N 5/06

(52) U.S. Cl. 514/12; 530/350; 435/69.1; 435/320.1; 435/325; 536/23.5

(57) ABSTRACT

The subject invention relates to peptide inhibitors of Akt as well as to uses of these inhibitors. More specifically, the inhibitors may be used, for example, to induce apoptosis in cells and sensitize tumor cells to cancer therapies. The peptides may also be used to purify Akt.

The subject application is a continuation-in-part of pending U.S. patent application Ser. No. 10/103,256 filed on Mar. 21, 2002, hereby incorporated in its entirety by reference.

### BACKGROUND OF THE INVENTION

1. Technical Field

The subject invention relates to peptide inhibitors of Akt as well as to uses of these inhibitors. More specifically, the inhibitors may be used, for example, to induce apoptosis in cells and sensitize tumor cells to cancer therapies. The peptides may also be used to purify Akt.

2. Background Information

Akt is a serine threonine protein kinase that is important to cell growth and survival. Three isoforms of Akt have been identified to date: Akt1, Akt2 and Akt3 [Datta et al., Genes and Development 13:2905-2927 (1999)].

With respect to the role of Akt in the kinase pathway, growth factors and survival factors initially activate PI3′ kinase. PI3′ kinase then phosphorylates PIP2 [PtdIns(4)P or PtdIns(4,5)P₂] into PIP3 [PtdIns(3,4)P₂ or PtdIns(3,4,5)P₃] which recruits PDK1, ILK1 and Akt to the plasma membrane. This facilitates the activation of Akt by PDK1 and ILK1. PTEN, on the other hand, is a PIP3 phosphatase that antagonizes the function of PI3′K and prevents Akt activation.

Activated Akt suppresses apoptosis by phosphorylating and inhibiting many target proteins that are required for apoptosis. In particular, Akt phosphorylates Bad protein and prevents Bad from binding to the mitochondrial membrane [Datta et al., Cell 91:231-41 (1997); del Peso et al., Science 278:687-689 (1997)]. Akt also phosphorylates and inactivates capsase 9 [Cardone et al., Science 282:1318-1321 (1998)]. In addition, Akt can phosphorylate and inactivate a forehead transcription factor that facilitates the expression of cell death factors such as Fas ligand [Brunet et al., Cell 96:857-868 (1999)]. Furthermore, Akt also phosphorylates IKKα, one of the subunits of the IκB kinase. The activated IKK phosphorylates IκB and targets it for degradation. This results in an increase in NFκB activity and the expression of proteins required for cell survival [Romashkova et al., Nature 401:86-90 (1999); Ozes et al., Nature 401:82-82 (1999)].

Relevance of Akt to tumorigenesis has previously been proposed, as mutations in PTEN have been frequently found in cancer patients. In fact, PTEN is the second most frequently mutated gene in malignancies. In contrast to the status of PTEN in tumors, Akt is often upregulated or constitutively activated in cancers.

In particular, Akt1 is constitutively active in all PTEN defective tumor cells. Akt2 is upregulated in 10-20% of pancreatic and ovarian cancers. Also, antisense of Akt2 has also been shown to prevent invasiveness of ASPC1 cells and PANC1 cells in a tracheal xenotransplant assay [Cheng et al., PNAS 93:3636-3641 (1996)]. Further, Akt3 is overexpressed in malignant breast and prostate cancer cell lines derived from advanced cancers [Nakatani et al., J Biol. Chem. 274:21528-21532 (1999)]. In addition, overexpression of constitutively active Akt1, or Akt2, or Akt3 can transform cells and induce tumorigenesis [Mirza, et al., Cell Growth & Differentiation 11:279-292 (2000); Cheng, et al., Oncogene 14:2793-801 (1997); Segrelles et al., Oncogene 21:53-64 (2002); Mende et al., Oncogene 20:4419-4423 (2002)].

Inhibition of Akt can block cell transformation. Oncogenic Ras can transform cells synergistically together with c-myc. C-myc induces faster cell growth and apoptosis. Oncogenic Ras suppresses the c-myc-induced apoptosis through the PI3′K-Akt pathway. A dominant negative mutant of Akt1 suppresses these transforming properties of oncogenic Ras [Kauffmann-Zeh et al., Nature 385:544-548 (1997)]. This dominant negative Akt1 has been also shown to block the transformation by oncogenic BCR/ABL and p3k (the constitutively active PI3K) [Skorski et al., EMBO Journal 16:6151-6161 (1997); Aoki et al., PNAS 95:14950-14955]. Recently, the activation of PI3K-Akt pathway has also been implicated in hypoxia response of the tumors and endothelial cell survival [Zundel et al., Genes & Development 14:391-396 (2000); Kim et al., Oncogene 19: 4549-52 (2000)].

One of the challenges in cancer therapy is to overcome drug resistance. Akt inhibitors can block the antiapoptotic pathway in tumor cells. Thus, Akt inhibitors may induce apoptosis in cells and sensitize tumor cells to cancer therapies. Consequently, such inhibitors may provide a much needed therapy to cancer patients.

All U.S. patents, patent applications and publications are herein incorporated in their entirety by reference.

### SUMMARY OF THE INVENTION

The subject invention encompasses amino acid or peptide sequences which bind to the substrate binding site of Akt (e.g., Akt1, Akt2 or Akt3, preferably Akt1), thereby preventing the activity thereof. These sequences are represented by or comprise SEQ ID NO: 1 (peptide 1), SEQ ID NO: 2 (peptide 2), SEQ ID NO: 3 (peptide 3), SEQ ID NO: 4 (peptide 4) and SEQ ID NO: 5 (peptide 5). The present invention also includes fragments of these sequences that have the functional activity of any one of more of these full-length peptides (i.e., bind to the substrate binding site of Akt and inhibit activity of Akt). The present invention also includes amino acid sequences having at least 70%, preferably at least 80% and more preferably at least 90% amino acid identity to the amino acid sequences of SEQ ID NO: 1, SEQ ID NO: 2, SEQ ID NO: 3, SEQ ID NO: 4, SEQ ID NO: 5, or a fragment thereof. (All integers within the range of 70% to 100% are also considered to fall within the scope of the present invention.)

The subject invention also encompasses isolated nucleic acid sequences which encode the above-described peptides. The nucleic acid sequences may be represented by or comprise SEQ ID NO: 6 (i.e., the sequence encoding peptide 1), SEQ ID NO: 7 (i.e., the sequence encoding peptide 2), SEQ ID NO: 8 (i.e., the sequence encoding peptide 3), SEQ ID NO: 9 (i.e., the sequence encoding peptide 4), SEQ ID NO: 10 (i.e., the sequence encoding peptide 5), or a fragment thereof which specifically hybridizes to the complement of one of these sequences. Also encompassed by the present invention is an isolated nucleic acid sequence comprising a nucleotide sequence having at least 70% identity, preferably at least 80% identity, and more preferably at least 90% identity to the nucleotide sequences of SEQ ID NO: 6, SEQ ID NO: 7, SEQ ID NO: 8, SEQ ID NO: 9 or SEQ ID NO: 10, or to a fragment thereof which hybridizes to the complement of the sequence having the above-described percent identities. (All integers within the range of 70% to 100% are considered to fall within the scope of the present invention, also.)

Additionally, the present method includes a method of inhibiting the activity of Akt in a mammalian cell comprising the steps of exposing the mammalian cell to at least one peptide of the present invention or a fragment thereof, capable of binding to Akt, for a time and under conditions sufficient for a complex to form between the at least one peptide and Akt1, wherein complex formation is indicative of binding of the peptide to the substrate binding site of Akt1 and thus inhibition of the functional activity of the kinase.

The present invention also includes a method of screening a compound or composition, in question, for the ability to inhibit activation of Akt (e.g., Akt1, Akt2 or Akt3) comprising the steps of exposing a mammalian cell to the composition of interest (for example, by introduction of a vector comprising a nucleotide sequence which encodes one or more of the peptides or by introducing the peptide(s) directly into the cell) and measuring Akt activity (e.g., by presence of an enzymatic reaction by-product), a lack of activity indicating a composition having the ability to inhibit the activity of the kinase.

Also, the present invention encompasses a method of screening a composition, in question, for the ability to inhibit activity of Akt in vitro comprising the steps of exposing the kinase to the composition of interest as well as to a substrate upon which the kinase normally acts (e.g., for example, a biotinylated peptide such as biotin-EELSPFRGRSRSAPPNLWAAQR (SEQ ID NO: 12)) and then detecting the presence or absence of the product produced as a result of the enzyme reaction between the enzyme (i.e., Akt) and the substrate. Presence of the product indicates that the kinase is active and has acted upon the substrate. Thus, the composition of interest did not bind to the substrate binding site of the kinase. In contrast, lack of a product indicates that the composition bound to the substrate binding site of the enzyme, and that enzyme activity has been inhibited.

Additionally, the present invention encompasses a pharmaceutical composition comprising at least one peptide of the present invention or a homologue thereof which inhibits the function of the Akt kinase. The composition also contains a pharmaceutically acceptable carrier.

Furthermore, the present invention encompasses a method of sensitizing malignant cells to chemotherapy, in a patient in need of such treatment, comprising the step of administering to the patient an effective amount of the pharmaceutical composition or compositions described above.

Additionally, the present invention includes a method of purifying Akt from a mixture of compounds comprising the steps of attaching at least one peptide or fragment thereof to a solid phase, wherein the at least one peptide or fragment thereof comprises an amino acid sequence having at least 70% identity, preferably 80% identity, and more preferably at least 90% identity to an amino acid sequence selected from the group consisting of SEQUENCE ID NO: 1, SEQUENCE ID NO: 2, SEQUENCE ID NO: 3, SEQUENCE ID NO: 4 and SEQUENCE ID NO: 5; and exposing the mixture to the at least one attached peptide or fragment thereof, for a time and under conditions sufficient for Akt of the mixture to bind to the attached peptide or fragment thereof, thereby purifying Akt from the mixture. (Again, all integers within the range of 70% to 100% are also considered to fall within the scope of the present invention.) The solid phase may be, for example, microtiter wells, test tubes, polystyrene beads, magnetic beads, nitrocellulose strips, membranes and microparticles.

Furthermore, the present invention encompasses a method of determining the effects of Akt on a cell comprising the steps of exposing a first cell to at least one peptide comprising an amino acid sequence having at least 70% amino acid identity to an amino acid sequence selected from the group consisting of SEQUENCE ID NO: 1, SEQUENCE ID NO: 2, SEQUENCE ID NO: 3, SEQUENCE ID NO: 4 and SEQUENCE ID NO: 5, and comparing the phenotypical characteristics or phenotype of the first cell with a second cell which has not been exposed to the at least one peptide, the comparison elucidating the effects of Akt on the cell.

Additionally, the present invention encompasses a method of introducing at least one peptide inhibitor into a cell, wherein the at least one peptide inhibitor comprises an amino acid sequence having at least 70% amino acid identity to an amino acid sequence selected from the group consisting of SEQUENCE ID NO: 1, SEQUENCE ID NO: 2, SEQUENCE ID NO: 3, SEQUENCE ID NO: 4 and SEQUENCE ID NO: 5. The method comprises the steps of exposing the at least one peptide inhibitor to a membrane translocating peptide (MTP) for a time and under conditions sufficient for the at least one peptide inhibitor to fuse to the MTP and exposing said cell to said resulting fused peptide such that the at least one peptide inhibitor is introduced into the cell. The MTP may be, for example, poly-D-Arginine.

### BRIEF DESCRIPTION OF THE DRAWINGS

FIG. 1 represents the amino acid sequences of five peptide inhibitors (peptide 1=SEQ ID NO: 1, peptide 2=SEQ ID NO: 2, peptide 3=SEQ ID NO: 3, peptide 4=SEQ ID NO: 4 and peptide 5=SEQ ID NO: 5). In particular, the figure illustrates the portion of each inhibitor derived from human FKHRL1 and the portion from Obata et al., supra.

FIG. 2: FIG. 2A illustrates the binding of peptide 1 in the Akt1 model; FIG. 2B illustrates the binding of peptide 2 in the Akt1 model; and FIG. 2C illustrates the binding of peptide 3 in the Akt1 model.

FIG. 3: FIG. 3A illustrates Akt1 inhibition by the five Akt1 peptide inhibitors. FIG. 3B illustrates the IC50 of the five peptide inhibitors.

FIG. 4 illustrates the putative nucleotide sequences of the peptide inhibitors. (Peptide 1 is encoded by SEQ ID NO: 6. Peptide 2 is encoded by SEQ ID NO: 7. Peptide 3 is encoded by SEQ ID NO: 8. Peptide 4 is encoded by SEQ ID NO: 9, and peptide 5 is encoded by SEQ ID NO: 10.)

FIG. 5 illustrates the purity of various fractions obtained from an initial insect cell lysate, as measured by absorbance, subsequent to exposure to various buffers (see Example IV). In particular, Akt was eluted with buffer D at fraction 21.

FIG. 6 illustrates an analysis of the insect cell lysate and column fractions by sodium dodecyl sulfate polyacrylamide gel electrophoresis (see Example IV).

FIG. 7 illustrates electrospray mass analysis (ESI-MS) data obtained to confirm isolation of the intact Akt1 molecule (see Example IV).

FIG. 8 represents the minimal length requirement for the peptide inhibitors. Different lengths of peptide inhibitor 2 were synthesized. For some of them, an acetyl group was added to the amino terminal end to increase the stability for cellular assays. These peptides were tested in the Akt assay described herein.

FIG. 9 illustrates binding between Akt peptide inhibitors and Akt1. Fluorescence polarization was used as an indication of binding between the 10 nM Oregon-green labeled peptides and Akt1 wild type. The fluorescence polarization value was plotted vs. different concentrations of Akt1, and Kd was derived. A. The binding between peptide 2 and Akt1 requires ATP-MgCl₂ (filled square: binding in the presence of 1 mM ATP-MgCl₂; open circle: binding in the absence of ATP-MgCl₂). B. Kd measurements in the presence of 1 mM ATP-MgCl₂. Symbols for each peptide are shown in C. The data are representative of several experiments using Akt1. C. Kd for each peptide.

FIG. 10 illustrates that Akt peptide inhibitors inhibit Akt inside cells. A. Peptide inhibitors as indicated in each lane were delivered into Hela cells via BioPORTER agents. Two hours or four hours post-delivery, cell lysates were prepared and analyzed by immunoblotting using the indicated antibodies. B. Hela cells were incubated with 25 μM Fluo-dr-peptide 2 or Fluo-dr-peptide 5 for 5 hours. The cells were examined using fluorescent microscope. The top panel shows the bright fields and the bottom panel shows the fluorescent fields. C. Hela cells were incubated with Fluo-dr-peptides as indicated. Three hours after the incubation, cell lysates were prepared and analyzed by immunoblotting using the indicated antibodies. In both A and C, the upper panels show GSK3 phosphorylation. The lower panels show total GSK3 protein levels.

FIG. 11 illustrates that Akt peptide inhibitor 2 inhibits cell growth. Cells were plated on 96 well plates with 7500 cell/well for MiaPaCa-2 cells or 5000 cells/well for Hela cells 20 hours before the peptide incubation. The cells were incubated with either Fl-dr-pep 2 or Fl-dr-pep 5 at the indicated concentration for 48 hours. AlamarBlue assays were carried according to vender's instructions.

### DETAILED DESCRIPTION OF THE INVENTION

The subject invention relates to novel peptide inhibitors of Akt that may be used for several purposes including, for example, therapeutic purposes. In particular, such peptides may be used to inhibit function of Akt kinase, thereby inducing apoptosis in cells and sensitizing tumor cells to cancer therapies. Additionally, the peptide inhibitors may be used in the purification of Akt.

The Peptide Inhibitors of Akt

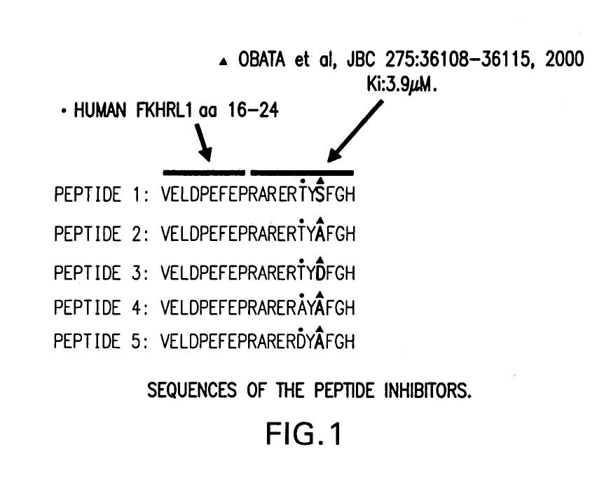

The amino acid sequences of the designed peptide inhibitors of Akt are shown in FIG. 1 (peptides 1-5). The present invention encompasses these sequences, fragments thereof, as well as amino acid sequences corresponding to having at least about 70%, preferably at least about 80%, and more preferably at least about 90% amino acid identity to one of the peptides of FIG. 1 (i.e., peptides 1-5). (All integers between the range of 70% to 100% are also considered to be within the scope of the present invention.) Furthermore, the present invention also includes fragments of these sequences as well.

For purposes of the present invention, a “fragment” of an amino acid sequence is defined as a contiguous sequence of approximately at least 11, preferably at least about 13, more preferably at least about 15, and even more preferably at least about 18 amino acids corresponding to a region of the specified peptide sequence.

Sequence identity or percent identity is the number of exact matches between two aligned sequences divided by the length of the shorter sequence and multiplied by 100. The algorithm of Smith and Waterman, Advances in Applied Mathematics 2:482-489 (1981), may be used with peptide or protein sequences using the scoring matrix created by Dayhoff, Atlas of Protein Sequences and Structure, M. O. Dayhoff ed., 5 Suppl. 3:353-358, National Biomedical Research Foundation, Washington, D.C., USA, and normalized by Gribskov, Nucl. Acids Res. 14(6):6745-66763 (1986). An implementation of this algorithm for peptide sequences is provided by the Genetics Computer Group (Madison, Wis.) in the BestFit utility application. The default parameters for this method are described in the Wisconsin Sequence Analysis Package Program Manual, Version 8 (1995) (available from Genetics Computer Group, Madison, Wis.). Other equally suitable programs for calculating the percent identity (or similarity) between sequences are generally known in the art. (For purposes of the present invention, “similarity” is defined as the exact amino acid to amino acid comparison of two or more polypeptides at the appropriate place, where amino acids are identical or possess similar chemical and/or physical properties such as charge or hydrophobicity. “Percent similarity” is calculated between the compared polypeptide sequences using programs known in the art (see above).

Functional equivalents of the above-sequences (i.e., sequences having the ability to bind to the substrate binding site of Akt and thus inhibit function of the Akt kinase or related kinases) are also encompassed by the present invention.

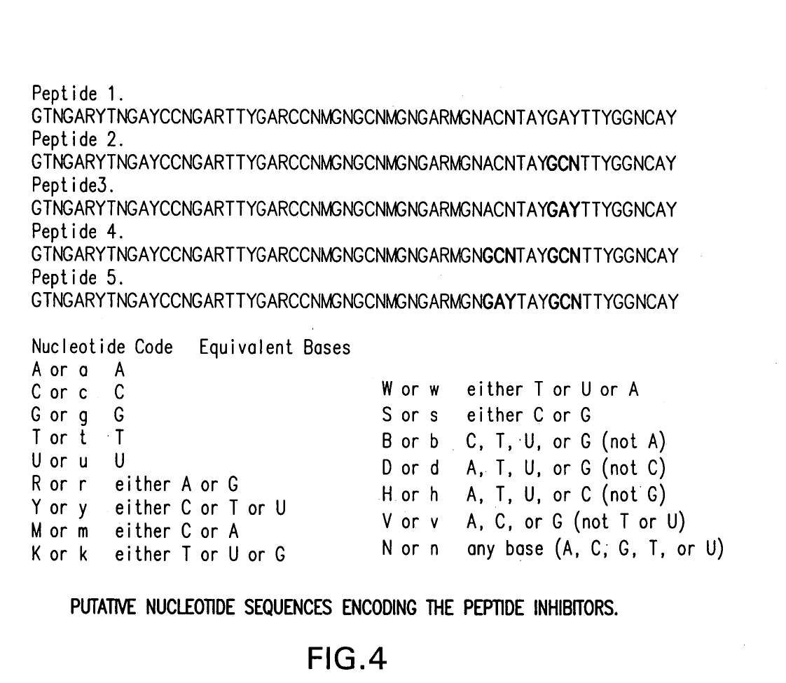

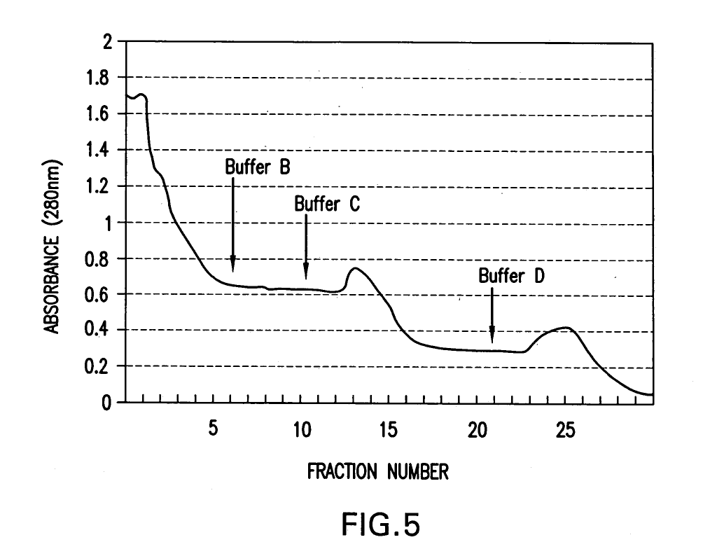

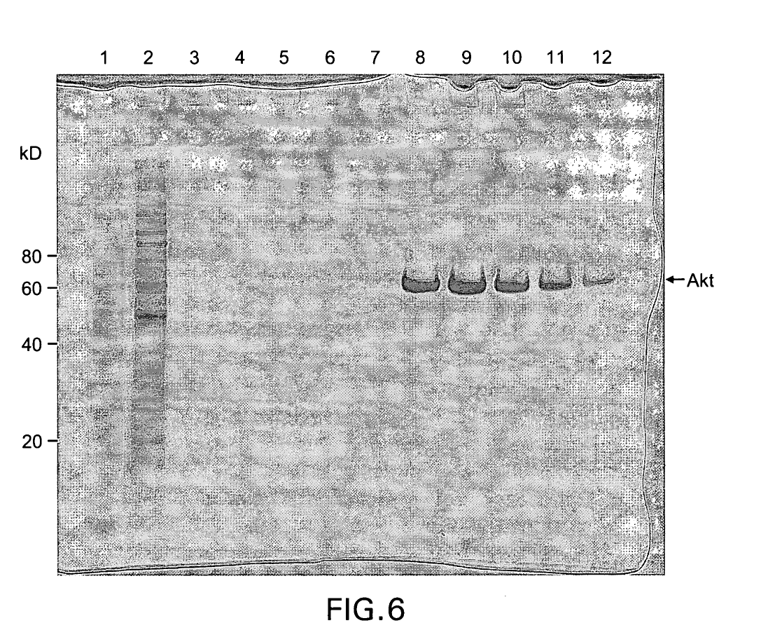

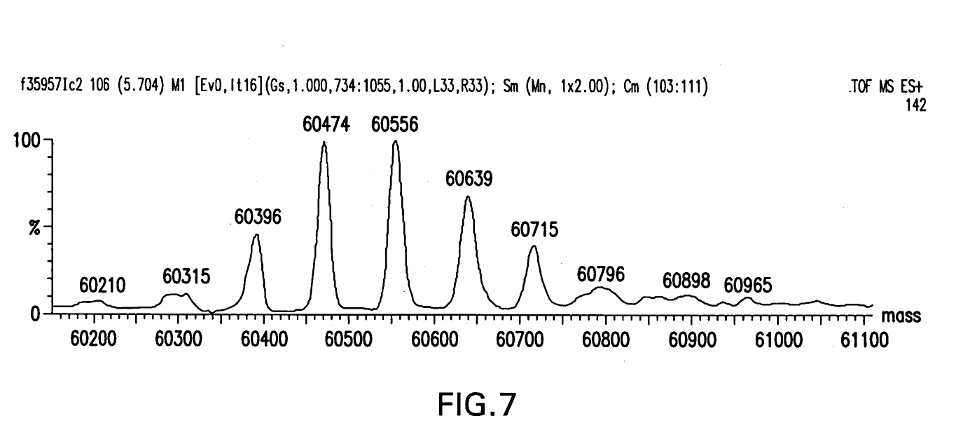

The present invention also includes isolated nucleotide sequences which encode the above-described peptides, as shown in FIG. 4. In particular, the present invention encompasses these sequences, fragments thereof, complements of the sequences and fragments, as well as sequences corresponding to (i.e., having identity to) or complementary to at least about 70%, preferably at least about 80%, and more preferably at least about 90% of the nucleotides in FIG. 4. (Again, all integers within the range of 70% to 100% are considered to fall within the scope of the present invention.) Furthermore, the present invention also includes fragments and complements of these sequences as well.

For purposes of the present invention, a “fragment” of a nucleotide sequence is defined as a contiguous sequence of approximately at least 6, preferably at least about 8, more preferably at least about 10 nucleotides, and even more preferably at least about 15 nucleotides corresponding to a region of the specified nucleotide sequence.

Furthermore, for purposes of the present invention, a “complement” is defined as a sequence which pairs to a given sequence based upon base-pairing rules. For example, a sequence A-G-T in one nucleotide strand is “complementary” to T-C-A in the other strand.

An approximate alignment for nucleic acid sequences is provided by the local homology algorithm of Smith and Waterman, Advances in Applied Mathematics 2:482-489 (1981), described above. An implementation of this algorithm for nucleic acid sequences is provided by the Genetics Computer Group (Madison, Wis.) in the BestFit utility application. The default parameters for this method are described in the Wisconsin Sequence Analysis Package Program Manual, Version 8 (1995) (available from Genetics Computer Group, Madison, Wis.). Other equally suitable programs for calculating the percent identity or similarity between sequences are generally known in the art.

Such nucleotide sequences may be derived from mammalian (e.g., human, rodent, etc.) as well as non-mammalian sources (e.g., bacterial, viral, etc.) and are also covered by the present invention. Functional equivalents of the above-sequences (i.e., sequences having the ability to encode peptides which inhibit the activity of Akt and which bind to the substrate binding site of Akt), are also encompassed by the present invention, as well as sequences which hybridize to the complement of the above-described nucleotide sequences.

A nucleic acid molecule is “hybridizable” to another nucleic acid molecule when a single-stranded form of the nucleic acid molecule can anneal to the other nucleic acid molecule under the appropriate conditions of temperature and ionic strength (see Sambrook et al., “Molecular Cloning: A Laboratory Manual, Second Edition (1989), Cold Spring Harbor Laboratory Press, Cold Spring Harbor, N.Y.). The conditions of temperature and ionic strength determine the “stringency” of the hybridization. “Hybridization” requires that two nucleic acid sequences contain complementary sequences. However, depending on the stringency of the hybridization, mismatches between bases may occur. The appropriate stringency for hybridizing nucleic acids depends on the length of the nucleic acids and the degree of complementation. Such variables are well known in the art. More specifically, the greater the degree of similarity or homology between two nucleotide sequences, the greater the value of Tm for hybrids of nucleic acids having those sequences. For hybrids of greater than 100 nucleotides in length, equations for calculating Tm have been derived (see Sambrook et al., supra). For hybridization with shorter nucleic acids, the position of mismatches becomes more important, and the length of the oligonucleotide determines its specificity (see Sambrook et al., supra).

Transfection of the Sequence(s) into a Host Cell and Expression of the Peptide(s)

One or more of the above-described nucleotide sequences, or fragments thereof, may be introduced into either a prokaryotic or eukaryotic host cell through the use of one or more transfection reagents. The transfection reagent or vector, for example, a bacteriophage, cosmid or plasmid, may comprise the full length nucleotide sequences described above or a fragment thereof, as any regulatory sequence (e.g., promoter) which is functional in the host cell and is able to elicit expression of the peptide or protein encoded by the nucleotide sequence. The regulatory sequence is in operable association with or operably linked to the nucleotide sequence. (A regulatory sequence (e.g., promoter) is said to be “operably linked” with a coding sequence if the promoter affects transcription or expression of the coding sequence.) Suitable regulatory sequences include, for example, CMV-based promoters (including but not limited to tetracyclin-regulated CMV promoters), the ecdysone-responsive promoter and the 5′ LTR, for expression in mammalian cells, GL4 (galactose inducible) and ADH1, for expression in yeast, and T7, T3, Sp6 and Lac, for expression in bacteria.

Additionally, other nucleotide sequences may also be included within the vector as well as other regulatory sequences, for example, a replication origin which maintains the vector in the cells after dividing and/or an antibiotic resistance gene (e.g., an ampicillin resistance gene) which confers antibiotic resistance. The choice of sequences present in the construct or vector is dependent upon the desired expression product or products as well as the nature of the host cell.

Once the vector has been constructed, it may then be introduced into the host cell of choice (e.g., eukaryotic or prokaryotic) by methods known to those of ordinary skill in the art including, for example, transfection, transformation and electroporation (see Molecular Cloning: A Laboratory Manual, 2^{nd} ed., Vol. 1-3, ed. Sambrook et al., Cold Spring Harbor Press (1989)). Suitable examples of eukaryotic host cells include, for example, mammalian cells (e.g., human, rat and murine cells) and yeast cells. Human cells include, for example, primary cells (e.g., fibroblasts), immortalized cell lines (e.g., 184B5), and tumor cell lines (e.g., NCI-H1299, Hela cells, HCT116, MCF7, PC-3, A431 and SW684). Rat cells include, for example, primary cells, immortalized cell lines, and tumor cell lines (e.g., Matlylu). Mouse cells include, for example, primary cells, immortalized cells lines (e.g., NIH3T3). Suitable yeast cells include, for example, Saccharomyces spp. (e.g., S. cerevisiae) and Candida spp. (e.g., C. albicans).

Expression in a host cell can be accomplished in a transient or stable fashion. Transient expression can occur from introduced constructs which contain expression signals functional in the host cell, but which constructs do not replicate and rarely integrate in the host cell, or where the host cell is not proliferating. Transient expression also can be accomplished by inducing the activity of a regulatable promoter operably linked to the gene of interest, although such inducible systems frequently exhibit a low basal level of expression. Stable expression can be achieved by introduction of a construct that can integrate into the host genome or that autonomously replicates in the host cell. Stable expression of the gene product of interest can be selected for through the use of a selectable marker located on, or transfected with, the expression construct, followed by selection for cells expressing the marker. When stable expression results from integration, the site of the construct's integration may occur randomly within the host genome or can be targeted through the use of constructs containing regions of homology with the host genome sufficient to target recombination with the host locus. Where constructs are targeted to an endogenous locus, all or some of the transcriptional and translational regulatory regions can be provided by the endogenous locus.

Additionally, it should be noted that the peptides of the present invention may not only be produced recombinantly. They may also be produced synthetically using standard methods known in the art.

Uses of the Peptide Inhibitors of Akt

The uses of the peptide inhibitors of the present invention are many. For example, the peptides, as noted above, may be introduced into a cell by a vector, for example, in order to prevent the Akt substrate from binding to the substrate binding site of Akt (i.e., Akt1, Akt2 or Akt3, preferably Akt1) and thereby preventing the Akt from acting as an enzyme upon the substrate. Tumor cells often have either overexpressed of constitutively active Akt. These Akt activities have been shown to prevent cell death in these tumor cells when treated with chemotherapeutic agents. Inhibiting Akt with peptide inhibitors may block the antiapoptopic pathway in tumor cells, induce apoptosis and sensitize tumor cells to cancer therapies.

In particular, the peptides of the present invention, or fragments thereof, may also be used as pharmaceuticals that bind to Akt and thereby prevent enzymatic activity of the kinase. The pharmaceutical composition may comprise one or more of the peptides or fragments thereof as well as a standard, well-known, non-toxic pharmaceutically acceptable, carrier, adjuvant or vehicle such as, for example, phosphate buffered saline (PBS), water, ethanol, a polyol, a vegetable oil, a wetting agent, or an emulsion such as a water/oil emulsion. The composition may be either in a liquid or solid form. For example, the composition may be in the form of a tablet, capsule or injectible. The dosage of the composition as well as the form may be readily determined by one of ordinary skill in the art.

Also, the peptides may be used in order to screen for compositions, of interest, which inhibit Akt and for designing pharmaceuticals having the same purpose.

Additionally, the peptides of the present invention may be used to purify Akt kinases. This may be achieved by conjugating one or more of the peptides of the invention to a solid support (e.g., a bead, microtiter well, etc.), adding a mixture which contains the kinases for a time and under conditions sufficient for the conjugated peptide to form a complex with the kinase, and then causing the kinase to be released from the peptide(s). Specifically, the cell extract can be applied to a column with peptide inhibitor-conjugated resin. Akt will bind to the peptide resin in the presence of ATP. Other proteins will be washed away from the resin in the presence of the ATP. When ATP is washed away from the resin later in time, Akt is released since the binding between the peptide inhibitors and Akt requires ATP.

As an example, the peptide may form a high affinity ternary complex with magnesium-adenosine triphosphate (Mg:ATP) and Akt1 molecules. In order to release the complex, another solution which lacks Mg:ATP and contains ethylenediamine tetraaetic acid (EDTA) (e.g., 1 mM) and arginine (e.g., 200 mM) is added. The EDTA and arginine act as releasing agents.

The peptides may also be utilized to aid in the crystallization of Akt proteins. In particular, it has been shown that a peptide inhibitor (PKI) of protein kinase A facilitates the crystallization of protein kinase A. Thus, it is thought that peptide inhibitors of Akt may also aide in the crystallization thereof. Such crystallization involves pre-incubation of the purified Akt with small molecule inhibitors, with Mg:ATP mimetics, or with Mg:ATP. This is followed by the addition of a 1:1 molar ratio of Akt inhibitory peptide. Excipients generally known to promote crystallization are subsequently added to the mixture.

Additionally, the peptide inhibitors of the present invention may be used to probe for the specific function of Akt inside a cell. In particular, one may transfect at least one peptide inhibitor into a cell and observe the resulting cellular phenotype. One may then compare the properties of the non-transfected or control cell with the properties of the transfected cell, and thereby determine the precise effects that Akt has on a functioning cell.

Method of Purification of Akt

As noted above, the peptide inhibitors of the present invention may also be used to purify Akt kinases. In particular, the specificity of the Akt binding site for one of the peptides is quite advantageous in terms of purification of Akt. The high selectivity and high affinity of the peptides for the kinase are also quite advantageous.

In connection with such purification, one may use several different formats. For example, one may covalently couple the peptide to a solid phase, for example, walls of wells of a reaction tray, test tubes, polystyrene beads (i.e., a beaded matrix), magnetic beads, nitrocellulose strips, membranes and microparticles (see U.S. Pat. No. 6,051,374). Preferably, a beaded-matrix is utilized. As will be described in detail below, the peptide-bead matrix may then be used to purify the kinase from a complex mixture of other proteins for the purpose of preparing homogeneous samples of Akt that may be used, for example, for protein crystallization and structure-based drug design. Additionally, the process may be applied to purification of other protein kinases in the same family to which Akt belongs.

One advantage of this process is that it permits purification of the kinase without the addition of extra amino acid affinity tags (e.g., glutathione S-transferase-tag ((GST)-tag) or 6×-his-tag) at the N-terminal or C-terminal of the protein. Such tags may interfere with crystallization. Additionally, the affinity process selects correctly folded, active Akt molecules by virtue of binding to the active site of the enzyme. Molecules that are not properly folded or active will not bind to the peptide-affinity matrix.

It should also be noted that peptides similar to those of the present invention may be designed and used to purify other kinases by conjugating them to a solid phase. The conjugated peptide(s) will preferentially bind the kinase of interest allowing separation from other components of a mixture.

The present invention may be illustrated by the use of the following non-limiting examples:

### EXAMPLE I

### Design of the Hybrid Peptides

Obata and colleagues published an optimal peptide sequence, ARKRERTYSFGHHA (AKTide-2T), that binds to the substrate binding site of mouse Akt1 and inhibits it with a Ki=12 μM (J. Biol. Chem. 275:36108-36115 (2000)). To achieve more potent peptide inhibitors of Akt1, a hybrid with amino acid 16-24 of human FKHRL1 (Genbank accession number AF03285)and the peptide AKTide-2T was designed and synthesized chemically with the putative phosphorylation site Serine at position 17 (FIG. 1)(see Example IV). In order to explore the relative affinity of the nonphosphorylatable substrate and the product of the reaction, the putative phosphorylation site Serine 17 in the peptide was changed to either Alanine or Aspartate to give rise to two other peptides, peptide 2 and peptide 3 (FIG. 1).

### EXAMPLE II

### Molecular Model of Peptides Binds to Human Akt1

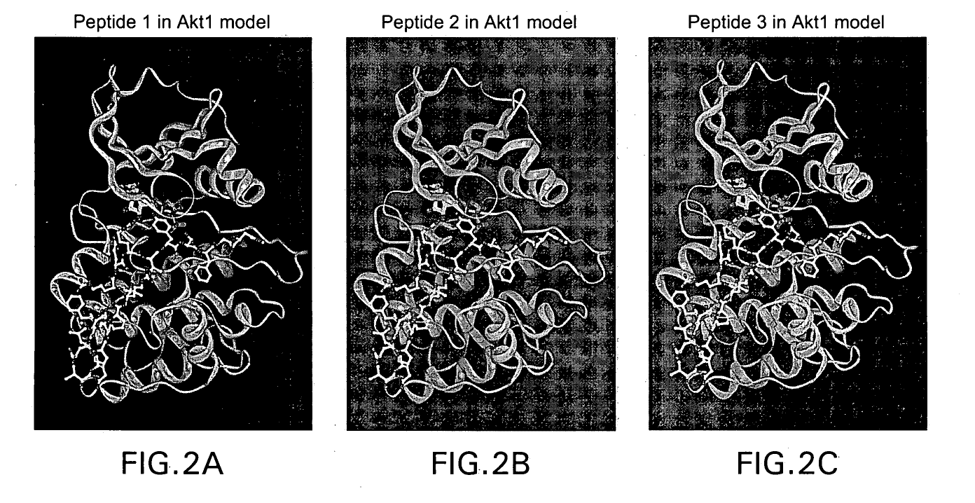

The molecular model of these peptides binds to human Akt1, as shown in FIG. 2. In particular, all of the peptides fit into the substrate binding site of Akt quite well. Substitution of the Serine with Alanine caused a change such that the peptide is small enough to fit into the substrate binding cleft. However, the Ser→Asp mutation in peptide 3 would interfere with the binding compared to the other two peptides.

### EXAMPLE III

### Inhibition of Akt1 in vitro

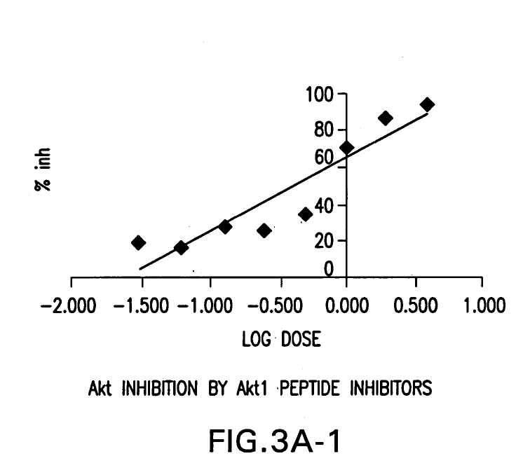

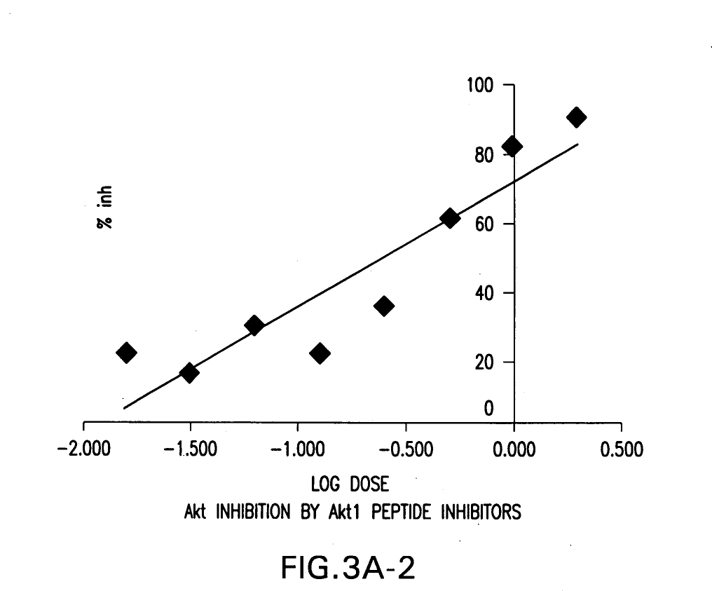

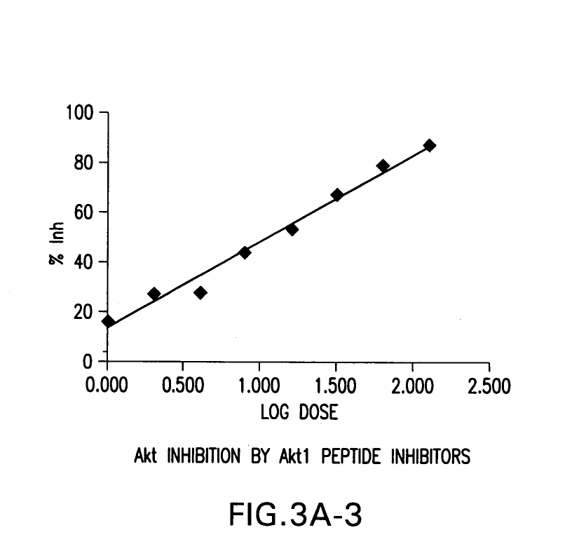

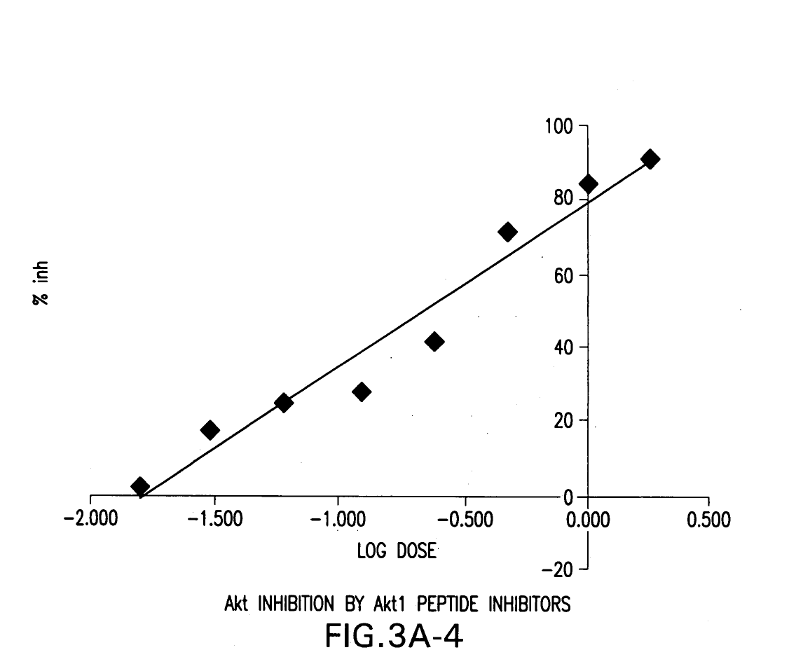

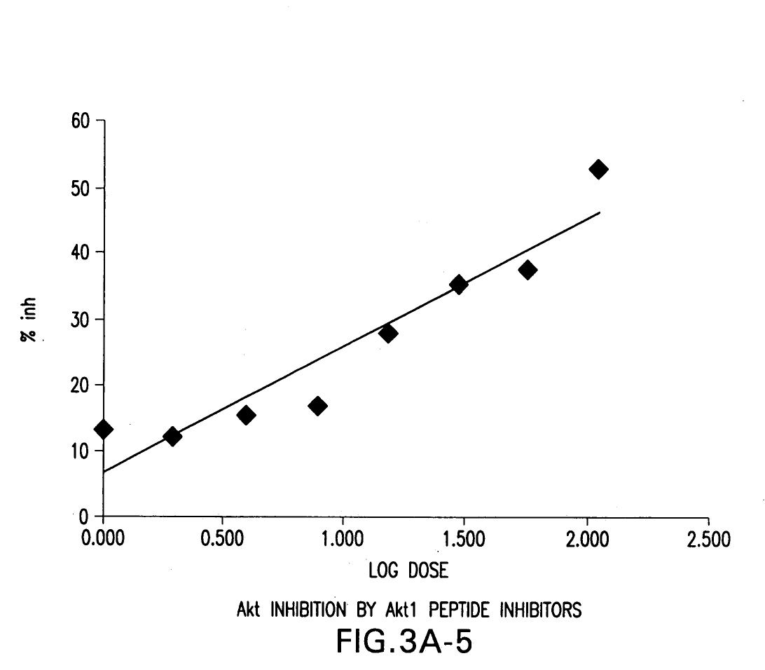

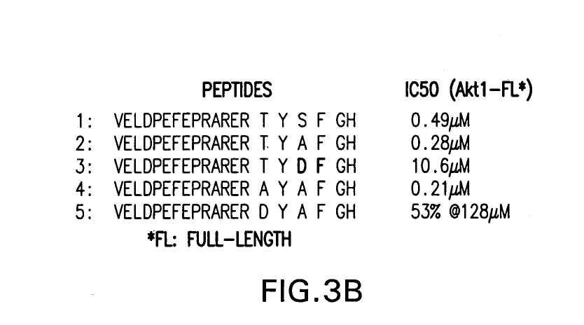

The peptides of the present invention were tested for their ability to inhibit Akt1 kinase activity in an in vitro kinase assay that utilizes a biotinylated peptide, Biotin-EELSPFRGRSRSAPPNLWAAQR, as a substrate. This peptide is derived from mouse Bad protein (Genbank Accession # A55671). The kinase assay was carried out under the following conditions: 20 mM HEPES, pH7.5,10 mM MgCl₂, 0.1% Triton X-100, 5 μM ATP (Km=40 μM), 5 μM Peptide (Km=15 μM). The inhibition curves by the five peptides are shown in FIG. 4A. Peptide 1, with Ser→Ala mutation at the phosphorylation site, is the most potent inhibitor against Akt1, indicated by the highest affinity (see FIG. 3). This is probably due to the fact that Akt1 binds the peptide but cannot phosphorylate it at the amino acid that has the Ser→Ala mutation. Thus, Akt1 cannot release it. Peptide 3 with the Ser→Asp mutation is the least potent possibly because of the interference of binding, as illustrated by the model (see FIG. 2C).

The three peptides described above also have a second putative Akt1 phosphorylation site at threonine (position 15). Thus, the Thr was mutated to Ala to form peptide 4 or Asp to form peptide 5 from peptide 2 (FIG. 1). These peptides should therefore bind to Akt in a similar fashion as peptide 1, peptide 2 and peptide 3. The IC50s were measured as before (FIG. 4), and similar results were obtained. Peptide 4 is as potent as peptide 2, and peptide 5 is the least potent inhibitor (see FIG. 3).

### EXAMPLE IV

### Peptide Synthesis and Preparation of Affinity Supports For Purification of Akt1

Materials:

All reagents were used as obtained from the vendor unless otherwise specified. Peptide synthesis reagents including diisopropylethylamine (DIEA), N-methylpyrrolidone (NMP), dichloromethane (DCM), 2-(1-H-benzotriazole-1-yl)-1,1,3,3-tetramethyluronium hexafluorophosphate (HBTU), N-hydroxybenzotriazole (HOBt), and piperidine were obtained from Applied Biosystems, Inc. (ABI), Foster City, Calif. Standard 9-Fluorenylmethyloxycarbonyl (Fmoc) amino acid derivatives (Fmoc-Ala—OH, Fmoc-Cys(Trt)—OH, Fmoc-Asp(tBu)—OH, Fmoc-Glu(tBu)—OH, Fmoc-Phe—OH, Fmoc-Gly—OH, Fmoc-His(Trt)—OH, Fmoc-Ile—OH, Fmoc-Leu—OH, Fmoc-Lys(Boc)—OH, Fmoc-Met—OH, Fmoc-Asn(Trt)—OH, Fmoc-Pro—OH, Fmor-Gln(Trt)—OH, Fmoc-Arg(Pbf)—OH, Fmoc-Ser(tBu)—OH, Fmoc-Thr(tBu)—OH, Fmoc-Val—OH, Fmoc-Trp(Boc)—OH, Fmoc-Tyr(tBu)—OH) were obtained from SynPep, Dublin, CA or ABI. Fmoc-amino acid resins (Fmoc-Ile-Wang, Fmoc-His(Trt)-Wang) were obtained from Novabiochem, San Diego, Calif. Trifluoroacetic acid (TFA), thioanisole, phenol, triisopropylsilane (TIS), ethanedithiol (EDT), acetic acid, and methanol was obtained from Acros/Fisher Scientific, Fair Lawn, N.J. Anhydrous isopropanol, ethanolamine and anhydrous dimethylsulfoxide (DMSO) were obtained from Aldrich Chemical Co., Milwaukee, Wis. Matrix-assisted laser desorption ionization mass-spectra (MALDI-MS) were recorded on an Applied Biosystems Voyager DE-PRO MS (Applied Biosystems, Inc., Foster City, Calif.). Electrospray mass-spectra (ESI-MS) were recorded on Finnigan SSQ7000 (Finnigan Corp., San Jose, Calif.) in both positive and negative ion mode.

General Procedure for Solid-Phase Peptide Synthesis (SPPS):

Peptides were synthesized with at most 250 μmol preloaded Wang resin/vessel on an ABI 430A peptide synthesizer using 250 μmol scale Fastmoc™ coupling cycles. For coupling standard Fmoc-amino acids, preloaded cartridges containing 1 mmol reagent were used with single-coupling. When the synthesis was complete, the resin was washed with 3×DCM and 3×MeOH, and dried in vacuo to give the protected peptide resin.

General Procedure for Cleavage and Deprotection of Resin-bound Peptide:

The peptides were cleaved from the resin by shaking the resin for 3 h at ambient temperature in a cleavage cocktail consisting of 80% TFA, 5% water, 5% thioanisole, 5% phenol, 2.5% TIS, and 2.5% EDT (1 mL/0.1 g resin). The resin was removed by filtration, rinsed with 2×TFA, the TFA evaporated from the filtrates, the residue precipitated with ether (10 mL/0.1 g resin), recovered by centrifugation, washed with 2×ether (10 mL/0.1 g resin) and dried to give the crude peptide.

General Procedure for Purification of Peptides:

The crude peptides were purified on a Gilson preparative HPLC system running Unipoint® analysis software (Gilson, Inc., Middleton, Wis.) on a radial compression column containing two 25×100 mm segments packed with Delta-Pak™ C18 15 μm particles with 100 Å pore size eluted with one of the gradient methods listed below. One to two milliliters of crude peptide solution (10 mg/mL in 90% DMSO/water) was purified per injection. The peaks containing the product(s) from each run were pooled and lyophilized. All preparative runs were run at 20 mL/min with eluents as buffer A: 0.1% TFA-water and buffer B: acetonitrile.

Gradient 1: 0-5 min: 10% B; 5-50 min: 1%/min gradient up to 55% B; 50-51 min: linear gradient to 95% B; 51-53 min: 95% B; 53-54 min: return to 5% B; 54-56 min: 10% B.

General Procedure for Analytical HPLC:

Analytical HPLC was performed on a Hewlett-Packard 1050 series system with a diode-array detector and a Hewlett-Packard 1046A fluorescence detector running HPLC 3D ChemStation software version A.03.04 (Hewlett-Packard. Palo Alto, Calif.) on a 4.6×250 mm YMC column packed with ODS-AQ, 5 μm particles with 120 Å pore size eluted with one of the gradient methods listed below after preequilibrating at the starting conditions for 7 min. Eluents were buffer A: 0.1% TFA-water and buffer B: acetonitrile. The flow rate for all gradients was 1 mL/min.

Gradient 1A: 0-5 min: 10% B; 5-85 min: 1%/min gradient up to 90% B; 85-95 min: 95% B.

VELDPEFEPRARERTYAFGH (1): Fmoc-His(Trt)-Wang resin (0.44 g, 150 μmol) (Novobiochem, Laufesfingen, Switzerland) was extended using the general peptide synthesis procedure to give the protected resin-bound peptide (0.821 g, 89.2%). The resin was cleaved and deprotected using the general procedure to give the crude peptide 1 as a white solid (0.43 g, 103.9%). Crude peptide 1 (0.2 g) was HPLC purified using gradient 1 with collection based on absorbance at 260 nm. Two peaks were isolated and lyophilized, with the major peak giving 1 as a white solid (0.065 g, 32.5%); ESI-MS m/z=1221.4 [(M+2Na)²⁺], 807.5 [(M+3H)³⁺], 1208.7 [(M−2H)²⁻], 805.6 [(M−3H)³⁻].

TTYADFIASGRTGRRNAI (2): Fmoc-Ile-Wang resin (0.675 g, 250 μmol) was extended using the general peptide synthesis procedure to give the protected resin-bound peptide (1.204 g, 85.2%). The resin was cleaved and deprotected using the general procedure to give the crude peptide 2 as a white solid (0.46 g, 75.7%). Crude peptide 2 (0.2 g) was HPLC purified using gradient 1 with collection based on absorbance at 220 nm. Two peaks were isolated and lyophilized, with the major peak giving 2 as a white solid (0.056 g, 28.0%; ESI-MS m/z=1970.4 [M+H]⁺.

Affygel-10-VELDPEFEPRARERTYAFGH (3): Affygel-10-NHS (50 mL, 0.75 mmol, 1 equiv.) was placed in a filter tube, drained, rinsed with 5×50 mL anhydrous isopropanol and 2×50 mL anhydrous DMSO. The peptide 1 (0.086 mg, 0.030 mmol, 0.04 equiv.) was dissolved in 50 mL anhydrous DMSO containing DIEA (0.29 g, 0.4 mL, 2.25 mmol, 3 equiv.) and added to the washed resin. The mixture was shaken at ambient temperature for 16 h, drained, suspended in 50 mL 1 M ethanolamine in anhydrous DMSO at ambient for 1 h, drained, rinsed with 2×50 mL anhydrous DMSO, 5×50 mL anhydrous isopropanol, and the resin 3 left suspended in 50 mL isopropanol.

Affygel-10-VELDPEFEPRARERTYAFGH (4): Affygel-10-NHS (25 mL, 0.375 mmol, 1 equiv.) was placed in a filter tube, drained, rinsed with 5×25 mL anhydrous isopropanol and 2×25 mL anhydrous DMSO. The peptide 2 (0.0324 mg, 0.0134 mmol, 0.04 equiv.) was dissolved in 25 mL anhydrous DMSO containing DIEA (0.145 g, 0.2 mL, 1.125 mmol, 3 equiv.) and added to the washed resin. The mixture was shaken at ambient for 16 h, drained, suspended in 25 mL 1 M ethanolamine in anhydrous DMSO at ambient for 1 h, drained, rinsed with 2×25 mL anhydrous DMSO, 5×25 mL anhydrous isopropanol, and the resin 4 left suspended in 25 mL isopropanol.

### EXAMPLE V

### Process for Purification of Akt-1 using Peptide-Affinity Matrix

Akt wild-type recombinant protein was over-expressed in a bacculovirus/insect cell expression system. The amino acid sequence of the recombinant Akt containing 529 residues with a calculated mass of 60439 Da was as follows:

| | |
|---|---|
| MSPIDPMGHHHHHHGRRPASVAAGILVPRGSPGLDGICSTEEFTMSDVAIVKEGWLH | (SEQ ID NO:11) |
| KRGEYIKTWRPRYFLLKNDGTFIGYKERPQDVDQREAPLNNFSVAQCQLMKTERPRP | |
| NTFIIRCLQWTTVIERTFHVETPEEREEWTTAIQTVADGLKKQEEEEMDFRSGSPSD | |
| NSGAEEMEVSLAKPKHRVTMNEFEYLKLLGKGTFGKVILVKEKATGRYYANKILKKE | |
| VIVAKDEVAHTLTENRVLQNSRHPFLTALKYSFQTHDRLCFVMEYANGGELFFHLSR | |
| ERVFSEDRARFYGAEIVSALDYLHSEKNVVYRDLKLENLMLDKDGHIKITDFGLCKE | |
| GIKDGATMKTFCGTPEYLAPEVLEDNDYGRAVDWWGLGVVMYEMMCGRLPFYNQDHE | |
| KTFELILMEEIRFPRTLGPEAKSLLSGLLKKDPKQRLGGGSEDAKEIMQHRFFAGIV | |
| WQHVYEKKLSPPFKPQVTSETDTRYFDEEFTAQMITITPPDQDDSMECVDSERRPHF | |
| PQFSYSASSTA. | |

The insect cell pellet was suspended in lysis buffer containing protease inhibitors (Complete™ EDTA-free; Roche, Indianapolis, Ind.), 20 mM Tris, 20 mM potassium phosphate, 150 mM potassium chloride, 10% (w/v) glycerol, 1 mM dithiothreitol (DTT), 1% (w/v) 3-[(3-cholamidopropyl)dimethyl-ammonio]-1-propanesulfonate (CHAPS), pH7.4 and lysed using a microfluidizer (Microfluidics, Newton, Mass.). The lysate was centrifuged at 23975×g for 30 min at 4° C., and the resulting supernatant was decanted. The slowly stirring supernatant at 4° C. was made 2 mM in magnesium chloride and 1 mM in adenosine triphosphate by the addition of solids. The pH was raised to pH7.4 by dropwise addition of 0.1N sodium hydroxide. To this mixture was added 40 mL of Akt peptide affinity resin that had been pre-equilibrated in wash buffer A (20 mM Tris, 20 mM potassium phosphate, 10 mM potassium chloride, 10% (w/v) glycerol, 1 mM DTT, 1% (w/v) CHAPS, 2 mM magnesium choride, 1 mM ATP, pH7.4). The resin and lysate were stirred slowly for 16hours at 4° C. to promote binding between the peptide and Akt. The resin was captured by filtration through a coarse scintered glass filter using gently suction. The wet beads were transferred to a glass chromatography column (XK 2.6×20 cm, Amersham-Pharmacia Biotech, Piscataway, N.J.) using a stream of ice cold buffer A from a wash bottle. The column was fitted to a chromatography system (Biologic, Biorad, Hercules, Calif.) where the absorbance measured at 280 nm was washed to baseline using buffer A at a flow rate of 1 mL/min and 10 mL fractions were collected. The column was washed with additional buffers to remove non-specifically bound proteins with buffer B (20 mM Tris, 20 mM potassium phosphate, 10% (w/v) glycerol, 1 mM DTT, 1% (w/v) CHAPS, 2 mM magnesium choride, 0.1% ethylphenyl-polyethylene glycol (Nonidet™ P40; USB, Cleveland, Ohio), 1 mM ATP, pH7.4) beginning at fraction 7 and buffer C (20 mM Tris, 20 mM potassium phosphate, 20 mM potassium chloride, 10% (w/v) glycerol, 1 mM DTT, 0.1% (w/v) CHAPS, 2 mM magnesium chloride, 1 mM ATP, pH7.4 beginning at fraction 11. The Akt was eluted with buffer D (200 mM arginine, 2 mM EDTA, 20 mM Tris, 50 mM potassium chloride, 10% (w/v) glycerol, 1 mM DTT, 1 mM sodium azide and pH7.4) beginning at fraction 21. The analysis of the insect cell lysate and the column fractions was followed by sodium dodecyl sulfate polyacrylamide gel electrophoresis (Invitrogen, Carlsbad, Calif.). Lane 1 contains 15 uL of 10 kD ladder molecular weight standards (10, 20, 30, 40, 50, 60, 70, 80, 90 and 100 kD; Invitrogen, Carlsbad, Calif.), lane 2 contains 1 uL of insect cell lysate supernatant, lanes 3-12 contain each respectively 15 uL aliquots from column fractions 21-30. It is apparent that highly purified Akt1 eluted in fractions 25-30 (lanes 7-12).

Electrospray mass analysis (ESI-MS) confirmed the intact molecule had been isolated. The Akt has apparently also been post-translationally modified by as many as five phosphorylations. Each apparent phosphorylation is observed as an extra 80 Da mass. Thus, in addition to the expected mass of 60304 which is observed at 60315 Da, five additional peaks are observed each with plus 80 mass (60396, 1 phosphate; 60474, 2 phosphates; 60556, 3 phosphates; 60639, 4 phosphates; 60715, 5 phosphates).

### EXAMPLE VI

### Minimal Peptide Length and Kinase Selectivity Assays

The following materials and methods apply to the present example and those that follow below:

Materials:

Enzymes: Akt1 was expressed and purified using a baculoviral system. PKA was purchased from Panvera. CDC2, PKA delta and PKC gamma were purchased from Calbiochem, and SGK and Src were purchased from Upstate Biotechnology (Charlottesvilee, Va.).

Substrates: All substrates were custom synthesized by Genemed Synthesis, Inc. (San Francisco, Calif.) except the Src substrate, which was purchased from Promega (cat#V6480). The substrates had the general structure of Biotin-Ahx-peptide. The Akt assay used EELSPFRGRSRSAPPNLWAAQR. The PKA assay used kemptide LRRASLG. The CDC2 assay used PKTPKKAKKL. The SGK assay used RPRAATF. The PKC delta and gamma used neurogranin ERMRPRKRQGSVRRRV. Phosphotidylserine and dioleyl-sn-glycerol were purchased from Avanti Polar Lipids (Alabaster, Ala.). Strepavidin Flash Plates were purchased from Perkin Elmer Life Sciences (Boston, Mass.).

Methods:

Peptide synthesis: Peptides were assembled on an 430A automated synthesizer (Applied Biosystems, Foster City, Calif.) using standard Fastmoc™ deprotection/coupling cycles with preloaded Wang resin (0.1-0.25 mmol). Cartridges containing N^{α}-Fmoc-amino acids (1 mmol) with side-chain protection (Arg, 2,2,5,7,8-pentamethylchroman-6-sulfonyl; Asp and Glu: tert-butyl ester; Asn, Cys, Gln, and His, trityl; Lys and Trp, tert-butyloxycarbonyl; Ser, Thr, and Tyr, tert-butyl ether) were activated with O-benzotriazol-1-yl-N,N,N′,N′-tetramethyluronium hexafluorophosphate (1 mmol), 1-hydroxybenzotriazole (1 mmol) and diisopropylethylamine (2 mmol) in N-methylpyrrolidone (NMP). The activated amino acid was coupled for 30 min following removal of the N-terminal Fmoc group with 20% piperidine in NMP. N-terminal acetylation was accomplished with acetic acid (1 mmol) coupled as for the Fmoc-amino acids. The peptides were cleaved and deprotected by shaking with reagent K (trifluoroacetic acid:water:thioanisole:phenol:ethanedithiol:triisopropylsilane, 80:5:5:5:2.5:2.5) [King, 1990 #5197] for 3 h at ambient temperature. The crude peptides were recovered by precipitation with ether following evaporation under reduced pressure. The peptides were purified on a preparative HPLC running Unipoint® analysis software (Gilson, Inc., Middleton, Wis.) on a 25×200 mm radial compression column containing Delta-Pak® C₁₈ packing (Waters, Inc., Taunton, Mass.) with a flow rate of 20 mL/min. The peptides were eluted with a linear gradient of 0.1% trifluoroacetic acid/water and acetonitrile. Fractions containing the peptide were pooled and lyophilized. The purity of the final products was confirmed by reverse-phase analytical HPLC on a Hewlett-Packard 1050 series system with diode-array and fluorescence detection (Agilent Technologies, Palo Alto, Calif.) eluted with a linear gradient of 0.1% trifluoroacetic acid/water and acetonitrile on a 4.6×250 mm YMC ODS-AQ, 5 μm, 120 A column (Waters, Inc.). The identity of the products was confirmed by electrospray ionization mass-spectrography (ESI-MS) on a Finnigan SSQ7000 (Finnigan Corp., San Jose, Calif.), or matrix-assisted laser desorption ionization mass-spectrography (MALDI-MS) on a Voyager DE-PRO (Applied Biosystems).

Kd Measurement: Fluorescence polarization measurements were performed at 25° C. in either a polarizing spectrofluorimeter (Photon Technologies International, London, Ontario) using 0.35 mL in quartz cuvettes or in an Analyst polarizing spectrofluorimeter (LJL Biosystems, Sunnyvale, Calif.) using 0.15 mL per well in 96-well plates. Akt1 wild type was diluted to the indicated concentrations in buffer containing 10 nM Oregon-green labeled Akt inhibitory peptides which were diluted from 100 μM DMSO stock solutions and incubated at 25° C. for 30 min prior to measurements. The buffer contained 20 mM Tris, 100 mM KCl, 10% glycerol, 1 mM DTT, 0.4 mM ATP, 1 mM MgCl2, pH 7.4. Data was fitted by non-linear least squares to the logistic equation using Origin software (Microcal Software, Inc., Northhampton, Mass.).

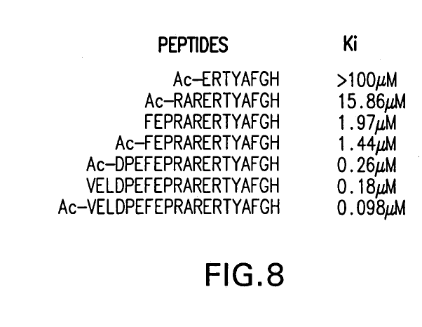

The minimal peptide length required for the maximal affinity was examined. A series of peptides were synthesized as described above, removing sets of 3 amino acids from the amino terminus. No significant change was observed when the peptide was shortened to 17 amino acids. However, any peptide less than 17 amino acids exhibited dramatically less affinity than the original 20 amino acid peptide. Therefore, 17 amino acids is the minimal length that gives highest affinity (see FIG. 8).

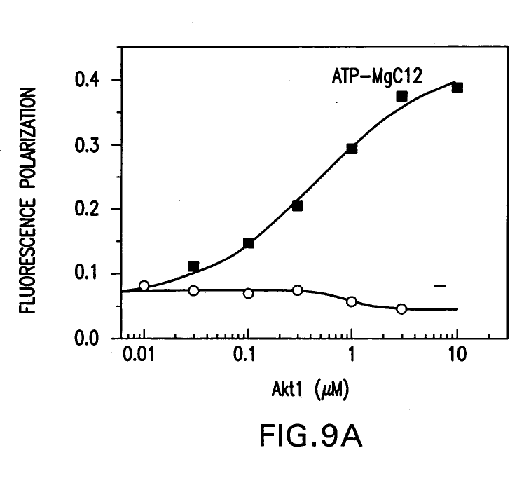

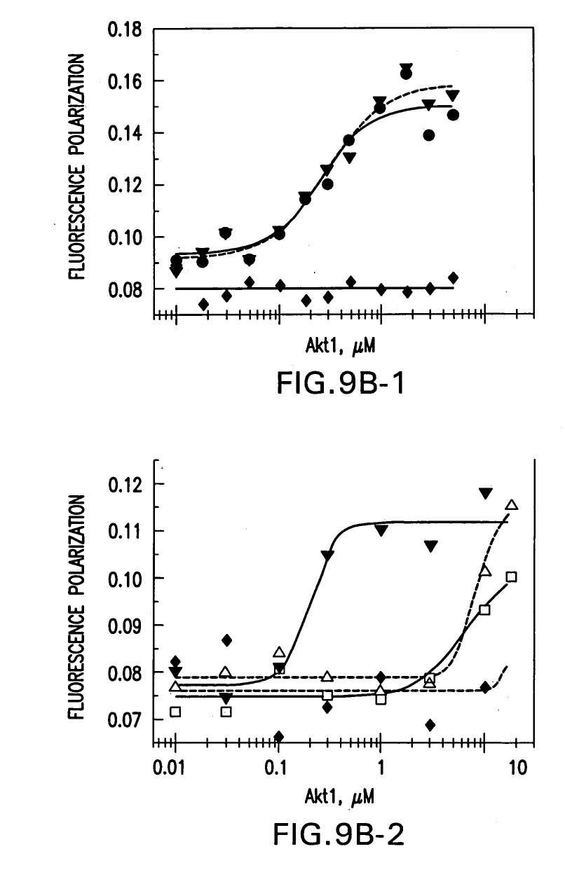

An examination was also performed relating to the requirements of the binding between the peptides and Akt. Fluorescence polarization was used as an indicator of binding between the Oregon-green labeled peptide and Akt1 protein. No binding was observed between peptide 2 and Akt1 in the absence of ATP-MgCl₂, while the Kd was measured to be 120 nM in the presence of ATP-MgCl₂ (FIG. 9A). This indicates that the binding requires ATP and suggests that Akt binds with ATP-MgCl₂ first before it binds with its substrate for phosphorylation. This is consistent with kinetic data from other kinases suggesting the reaction is ordered, and requires the first addition of ATP, prior to the protein substrate.

A measurement was also made relating to the binding constant between these peptides and Akt. An Akt1 titration experiment allowed measurement of the Kd of these peptides. FIG. 9 showed that the Kd of these peptides agrees very well with their potency in inhibiting Akt activity in vitro (FIGS. 9B and 9C).

Turning to the kinase selectivity assays, all the enzymes other than Akt were tested in 25 mM Hepes buffer pH 7.4, 10 mM MgCl₂, 0.1 mM Na₃VO₄, 0.5 mM DTT and 0.075 mg/ml Triton X-100. PKC gamma and PKC delta required 90 μg/ml phosphotidylserine and 18 μg/ml dioleyl-sn-glycerol. PKC gamma assay was run in the presence of 1 mM CaCl₂ and Src in the presence of 1 mM MnCl₂. PKA, CDC2, SGK and Src assay buffers contained 0.05% gelatin. Peptide inhibitors were dissolved in DMSO, tested at several concentrations, the highest of which was 50 μM. DMSO concentration in the assay was 2%. Enzymes, peptide substrates, ATP and γ-p33-ATP were in the following concentrations: 1. PKA assay: 0.44 nM enzyme, 3.5 μM peptide substrate, 7.5 μM ATP and 7.5 μCi/ml γ-p33-ATP, 2. CDC2 assay: 0.022 nM enzyme, 2.5 μM peptide substrate, 5 μM ATP and 20 μCi/ml γ-p33-ATP, 3SGK assay: 0.83 nM enzyme, 1 μM peptide substrate, 10 μM ATP and 10 μCi/ml γ-p33-ATP, 4. Src assay: 3.2 nM enzyme, 6 μM peptide substrate, 5 μM ATP and 5 μCi/ml γ-p33-ATP, 5. PKC gamma assay: 2.1 nM enzyme, 5 μM peptide substrate, 10 μM ATP and 10 μCi/ml γ-p33-ATP, 6. PKC delta assay: 2 nM enzyme, 5 μM peptide substrate, 10 μM ATP and 10 μCi/ml γ-p33-ATP. Reactions of 50 μl were carried out at room temperature for 30 min then stopped by the addition of 50 μl of 4 M NaCl/0.1 M EDTA pH 8.0. Reactions were transferred to Flash Plates, vortexed and incubated at room temperature for 10 min, then washed 3 times with phosphate buffered saline with 0.05% Tween 20 and counted on a TopCount Packard Instruments gamma counter (Packard Instruments, Boston, Mass.). IC50s were calculated using the Assay Explorer software, sigmoidal curve fit by MDL.

Each kinase has its own signature in the substrate-binding groove. Since the peptide inhibitors were designed to bind to the substrate binding site, it was expected that these peptides would be selective inhibitors of Akt. The peptides were tested against kinases that represent different classes. Indeed, no inhibition was observed on PKA, PKCδ, PKCγ, Cdc2 and Src, even though, PKA, PKCδ and PKCγ are within the AGC kinase family as is Akt (see attached Table 1). On the contrary, inhibition was observed on SGK by peptide 1. This is not a surprise to since SGK and Akt share many common downstream targets. However, peptide 2 or peptide 4 showed significant selectivity over SGK, suggesting the possibility of designing selective pseudo-substrate peptide inhibitors, even among the kinases that share some common substrates.

### EXAMPLE VII

### Delivery of Peptide Inhibitors into Cells

The peptide inhibitors were delivered into cells using the BioPORTER reagent (Gene Therapy Systems, San Diego, Calif.) according to the vendor's instructions. Specifically, the cells were washed with serum-free media once. The peptide solutions were used to hydrate the dried BioPORTER reagent. Five minutes after coincubation at room temperature, the peptide-BioPORTER complex was vortexed gently, mixed with serum-free media and added to the cells. Four hours after incubation, the medium was changed to the complete medium for further culture.

Poly-D-Agr: Hela cells were incubated with Flou-dr-peptide 2 in the complete medium.

Using a fluorescent-labeled peptide, the delivery efficiency was estimated to be over 90% when using BioPORTER reagents (data not shown).

### EXAMPLE VIII

### GSK3 Western Blot

Cells were harvested and lysed with brief sonication in insect cell lysis buffer (BD pharmingen, 10 mM Tris pH 7.5, 130 mM NaCl, 1% Triton X-100, 10 mM NaF, 10 mM NaPi, 10 mM NaPPi) with addition of 1X protease inhibitor cocktail (BD Pharmingen, San Diego, Calif., 16 μg/ml benzamidine HCl, 10 μg/ml phenanthroline, 10 μg/ml aprotinin, 10 μg/ml leupeptin, 10 μg/ml pepstatin A, 1 mM PMSF) and 1 μM microcystin LR (Sigma). Forty μg of total protein were loaded and electrophoresed under reducing conditions on NuPage 4-12% bis-tris gel (Invitrogen, Carlsbad, Calif.). Western blot was performed with phospho-GSK3a/b (ser21/9) antibody (Cell Signaling, Beverly, Calif.) (1:1000) GSK-3β antibody (Santa Cruz) (1:2000).

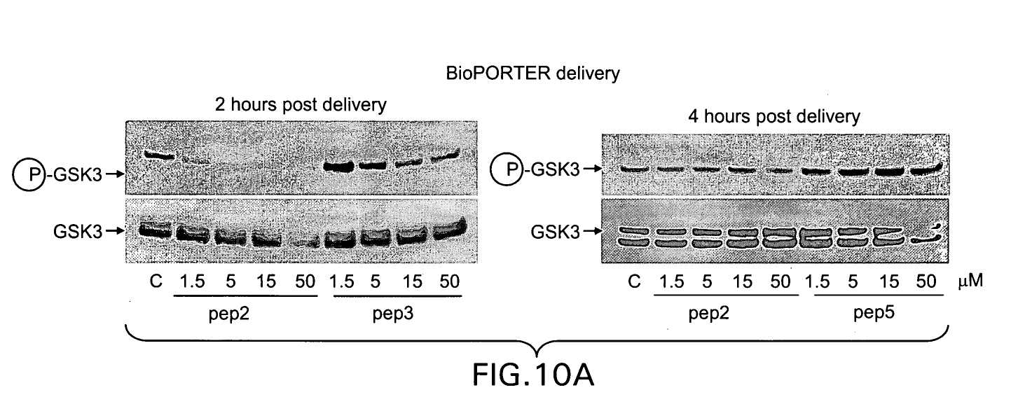

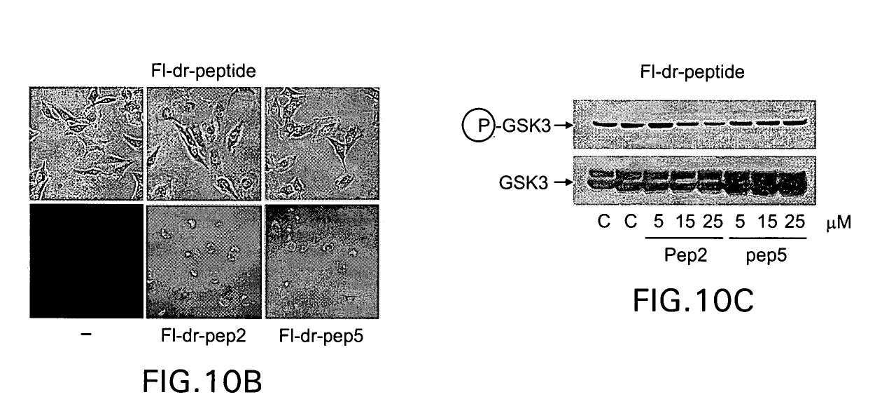

One of the major downstream phosphorylation targets of Akt is GSK3. Therefore, GSK3 phosphorylation was used as an indication of cellular Akt activity. Two hours after the delivery process, the total cell lysates were prepared and the GSK3 phosphorylation was assessed with GSK3-p antibody. Both peptide 2 and peptide 3 inhibited GSK3 phosphorylation in Hela cells in a dose dependent manner. Consistent with the in vitro kinase inhibition potency, peptide 2 was more potent than peptide 3 in inhibiting GSK3 phosphorylation. In the same experiment, total GSK3 protein levels were not changed by the peptide inhibitors (FIG. 10A).

Blocking Akt expression by antisense oligonucleotide induces cell growth inhibition and apoptosis in mammalian cells. Thus, an examination was made of the phenotype of cells after the delivery of these peptide inhibitors into Hela cells. Cell growth inhibition was not observed by peptide 2 or peptide 3 (data not shown). Studies with Akt small molecular inhibitors have demonstrated that a minimum of 16 hours of Akt inhibition is required for the maximal effect on cell growth inhibition (unpublished results). The peptide inhibitors may not be stable long enough to induced growth inhibition. Indeed, when an examination was made of the GSK3 phosphorylation at 4 hour post delivery, no reduction was observed by peptide 2, suggesting that peptide 2 is not stable after 4 hours in cells (FIG. 10A).

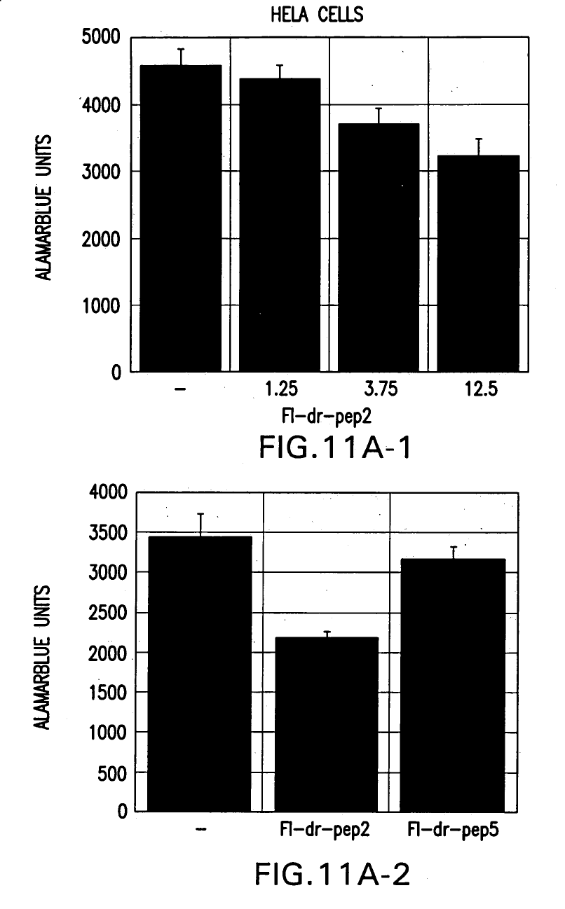

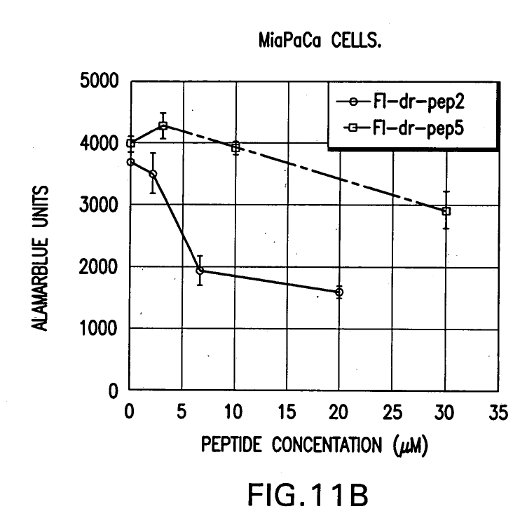

In order to achieve a continuous delivery of the peptide inhibitors into cells, poly-D-Arginine was used as a membrane translocating peptide (MTP) to facilitate the cell take-up of the peptide inhibitors. The fusion peptides contain a Fluorescent tag, poly-D-Arginine, and peptide inhibitors were synthesized. Fluorescent microscope examination indicated that more 90% of Hela cells or MiaPaCa cells took up the fusion peptides (FIG. 10B). A dose dependent inhibition on the GSK3 phosphorylation was also observed with Fl-dr-peptide 2 while Fl-dr-peptide 5 had no effect (FIG. 10C). In addition, significant growth inhibition was observed with Fl-dr-peptide 2 in both Hela and MiaPaCa cells, while no growth inhibition was induced by Fl-dr-peptide 5 (FIG. 11). This is consistent with the fact that continuous incubation of cells with Fl-dr-peptide2 would allow a partial but persistent Akt inhibition, which results in partial growth inhibition.

### EXAMPLE IX

### AlamarBlue Assay

The cells were gently washed with 200 μl of PBS. AlamarBlue reagent (Biosource International, Carmarillo, Calif.; Cat#: DAL11100) was diluted 1/10 in normal growth media. The diluted AlamarBlue reagent (100 μl) was added to each well on the 96-well plate and incubated until the reaction was complete as per manufacturer's instructions. Analysis was performed using a fmax Fluorescence Microplate Reader (Molecular Devices, Sunnyvalle, Calif.), set at the excitation wavelength of 544 nm and emission wavelength of 595 nm. Data was analyzed using SOFTmax PRO software provided by the manufacturer.

The results of the assay are presented in FIG. 11. In particular, the data shows that Akt peptide inhibitor 2 inhibits cell growth. More specifically, significant growth inhibition was observed with Fl-dr-peptide 2 in both Hela and MiaPaCa cells, while no growth inhibition was induced by Fl-dr-peptide 5. This is consistent with the fact that continuous incubation of cells with FL-dr-peptide 2 would allow for partial but persistent Akt inhibition, which results in partial growth inhibition.

TABLE 1
Selectivity of the peptide inhibitors.

| Ki | Akt 1 | PKA | Cdc2 | Src | SGK | PKCδ | PKCγ |
|---|---|---|---|---|---|---|---|
| Peptide 1 | 1.11 μM | >34 μM | >33 μM | >33 μM | 1 μM | >42 μM | >30 μM |
| Peptide 2 | 0.11 μM | >34 μM | >33 μM | >33 μM | >36 μM | >42 μM | >30 μM |
| Peptide 4 | 0.095 μM | >34 μM | >33 μM | >33 μM | 12.5 μM | >42 μM | >30 μM |

1. A purified peptide or a fragment thereof comprising an amino acid sequence having at least 70% amino acid identity to an amino acid sequence selected from the group consisting of SEQUENCE ID NO: 1, SEQUENCE ID NO: 2, SEQUENCE ID NO: 3, SEQUENCE ID NO: 4 and SEQUENCE ID NO: 5.

2. The purified peptide of claim 1 wherein said peptide or fragment thereof comprises an amino acid sequence selected from the group consisting of SEQUENCE ID NO: 1, SEQUENCE ID NO: 2, SEQUENCE ID NO: 3, SEQUENCE 4 and SEQUENCE ID NO: 5.

3. An isolated nucleotide sequence encoding said purified peptide or fragment thereof of claim 1 or 2.

4. An isolated nucleic acid sequence or fragment thereof comprising a nucleotide sequence having at least 70% identity to a nucleotide sequence selected from the group consisting of SEQUENCE ID NO: 6, SEQUENCE ID NO: 7, SEQUENCE ID NO: 8, SEQUENCE ID NO: 9 and SEQUENCE ID NO: 10.

5. The isolated nucleotide sequence or fragment thereof of claim 4, wherein said nucleotide sequence or fragment thereof comprises a sequence selected from the group consisting of SEQUENCE ID NO: 6, SEQUENCE ID NO: 7, SEQUENCE ID NO: 8, SEQUENCE ID NO: 9 and SEQUENCE ID NO: 10.

6. A method of inhibiting the function of Akt in a mammalian cell comprising the steps of exposing said cell to at least one peptide comprising an amino acid sequence having at least 70% identity to an amino acid sequence selected from the group consisting of SEQUENCE ID NO: 1, SEQUENCE ID NO: 2, SEQUENCE ID NO: 3, SEQUENCE ID NO: 4 and SEQUENCE ID NO: 5, for a time and under conditions sufficient for said at least one peptide to bind to Akt in order to form a complex, whereby said bound Akt is inhibited from functioning.

7. The method of claim 6 wherein said peptide comprises an amino acid sequence selected from the group consisting of SEQUENCE ID NO: 1, SEQUENCE ID NO: 2, SEQUENCE ID NO: 3, SEQUENCE ID NO: 4 and SEQUENCE ID NO: 5.

8. The method of claim 6 wherein said Akt is selected from the group consisting of Akt1, Akt2 and Akt3.

9. A method of screening a composition for the ability to inhibit activity of Akt comprising the steps of exposing a mammalian cell to said composition and measuring a reaction product of Akt activity, lack of said product indicating a composition having the ability to inhibit activity of Akt.

10. A method of screening a composition for the ability to inhibit activity of Akt comprising the steps of:
a) exposing Akt to said composition and to a substrate upon which Akt acts enzymatically; and
b) detecting presence or absence of the product produced as a result of enzymatic reaction between Akt and said substrate, absence of said product indicating that said Akt has not acted upon said substrate and has been inhibited by said composition.

11. A pharmaceutical composition comprising at least one peptide or fragment thereof of claim 1 and a pharmaceutically acceptable carrier.

12. A pharmaceutical composition comprising: 1) at least one purified peptide or fragment thereof comprising an amino acid sequence selected from the group consisting of SEQUENCE ID NO: 1, SEQUENCE ID NO: 2, SEQUENCE ID NO: 3, SEQUENCE ID NO: 4 and SEQUENCE ID NO: 5, and 2) a pharmaceutically acceptable carrier.

13. A method of sensitizing malignant cells to chemotherapy, in a patient in need of such treatment, comprising the step of administering to said patient an effective amount of the pharmaceutical composition of claim 11 or 12.

14. A method of inducing apoptosis in a cell comprising the steps of exposing said cell to at least one peptide or fragment thereof comprising an amino acid sequence having at least 70% amino acid identity to an amino acid sequence selected from the group consisting of SEQUENCE ID NO: 1, SEQUENCE ID NO: 2, SEQUENCE ID NO: 3, SEQUENCE ID NO: 4 and SEQUENCE ID NO: 5, for a time and under conditions sufficient for said at least one peptide or fragment thereof to bind to Akt, said binding inactivating Akt, said inactivation inducing said apoptosis in said cell.

15. The method of claim 14 wherein said at least one peptide or fragment thereof, exposed to said cells, comprises an amino acid sequence selected from the group consisting of SEQUENCE ID NO: 1, SEQUENCE ID NO: 2, SEQUENCE ID NO: 3, SEQUENCE ID NO: 4 and SEQUENCE ID NO: 5.

16. A method of purifying Akt from a mixture of compounds comprising the steps of:
a) attaching at least one peptide or fragment thereof to a solid phase, wherein said at least one peptide or fragment thereof comprises an amino acid sequence having at least 70% identity to an amino acid sequence selected from the group consisting of SEQUENCE ID NO: 1, SEQUENCE ID NO: 2, SEQUENCE ID NO: 3, SEQUENCE ID NO: 4 and SEQUENCE ID NO: 5; and
b) exposing said mixture to said at least one attached peptide or fragment thereof, for a time and under conditions sufficient for Akt of said mixture to bind to said attached peptide or fragment thereof, thereby purifying Akt from said mixture.

17. The method of claim 15 wherein said solid phase is selected from the group consisting of microtiter wells, test tubes, polystyrene beads, magnetic beads, nitrocellulose strips, membranes and microparticles.

18. A method of determining the effects of Akt on a cell comprising the steps of:
a) exposing a first cell to at least one peptide inhibitor comprising an amino acid sequence having at least 70% amino acid identity to an amino acid sequence selected from the group consisting of SEQUENCE ID NO: 1, SEQUENCE ID NO: 2, SEQUENCE ID NO: 3, SEQUENCE ID NO: 4 and SEQUENCE ID NO: 5; and
b) comparing the phenotypical characteristics of said first cell with a second cell which has not been exposed to said at least one peptide inhibitor, said comparison elucidating said effects of Akt on a cell.

19. A method of introducing at least one peptide inhibitor into a cell, wherein said at least one peptide inhibitor comprises an amino acid sequence having at least 70% amino acid identity to an amino acid sequence selected from the group consisting of SEQUENCE ID NO: 1, SEQUENCE ID NO: 2, SEQUENCE ID NO: 3, SEQUENCE ID NO: 4 and SEQUENCE ID NO: 5, comprising the steps of exposing said at least one peptide inhibitor to a membrane translocating peptide (MTP) for a time and under conditions sufficient for said at least one peptide inhibitor to fuse to said MTP and exposing said cell to said resulting fused peptide such that said at least one peptide inhibitor is introduced into said cell.

20. The method of claim 19 wherein said MTP is poly-D-Arginine.
